(19) United States
(12) Patent Application Publication
(10) Pub. No.: US 2003/0073941 A1
(43) Pub. Date: Apr. 17, 2003

(54) Method and apparatus for epicondylar protection

(76) Inventor: Jay E. Betz, Grass Valley, CA (US)

Correspondence Address: Herbert C. Schulze, 2790 Wrondel Way, PMB 36, Reno, NV, 89502, US

(21) Appl. No.: 10/278,766

(22) Filed: Oct. 21, 2002

Related U.S. Application Data
(63) Continuation-in-part of application No. 09/492,577, filed on Jan. 27, 2000, now abandoned.

(51) Int. Cl. A61F 5/00

(52) U.S. Cl. 602/5; 602/20

(57) ABSTRACT

A method and apparatus for use by individuals engaged in strenuous activities in which the apparatus is used to control and protect the muscles, ligaments and tendons in such manner that otherwise violent and uncontrolled motion of the hand and wrist and the like which would normally aggravate the epicondyle areas is restrained and protected and trained in such manner that motion of the hand and wrist becomes programmed in such manner that such motion is performed in a natural and complete restrained and controlled manner so to avoid aggravation of the epicondyle areas.

### CROSS REFERENCE TO RELATED PATENT APPLICATIONS

This application is related to, and is a continuation in part of, my application with the same title Ser. No. 09/492,577 filed Jan. 27, 2000.

### BACKGROUND OF THE INVENTION

I. Field of the Invention

This invention is in the general field of protection of body joints, bones, tendons and the like;

The invention is more particularly directed to the fields of protection of epicondylar areas of bones and their connection to muscles, tendons, ligaments, or the like;

The invention is most particularly in the field of relief of, and prevention of, pain in the epicondylar areas due to stress resulting from strenuous and somewhat violent bodily movements occurring while persons are engaged in vigorous sports and the like. The invention is uniquely and particularly directed to training and restraining violent wrist action in playing tennis and like activities to prevent tennis elbow.

II. Description of the Prior Art

The prior art in this field has been confined to the treatment and alleviation of discomfort from the symptoms resulting from such activities as are mentioned above. The present state of the art is virtually confined to compression distal to the epicondyle or the elbow. Additionally there have been a number of differing approaches, which have not been widely accepted. Recent examples are shown in the following U.S. patents: U.S. Pat. No. 4,364,564 reveals a system in which a quantity of mercury in a container is fastened to a wrist with the purpose of having the mercury absorb some of the vibration of striking a tennis ball or the like; U.S. Pat. No. 5,401,017 is a device which locks into position on a wrist and restrains movement until a certain point of impact at which it releases; U.S. Pat. No. 5,441,058 is a strap with special protruding areas to contact certain muscle masses with a view to relieve the symptoms of carpal tunnel syndrom and the like; U.S. Pat. No. 5,638,548 is a glove to be worn by an athlete which has means to restrain movement of the wrist; U.S. Pat. No. 5,566,389 is a glove-like wrist encompassing device with layered resilient materials and the like designed to restrain wrist movement; U.S. Pat. No. 5,891,079 is a sleeve-like device with elastic straps designed to relieve pain in the arm and elbow and to restrict movement.

With particular attention to the ailment commonly called “tennis elbow” I have studied other patents:

U.S. Pat. No. 5,472,412: This is a device which clamps over a knee joint onto the lower thigh and upper calf to limit the knee activity during rehabilitation and the like;

U.S. Pat. No. 5,252,102: This is a complex electronic Continuous Passive Motion apparatus in which the inventor sets forth the uses and purposes as “-a therapy machine that is used for gentle muscle stretching, or following surgery to rehabilitate the joint.-”;

U.S. Pat. No. 4,191,373: This device is more complex, but essentially using basically the same principles as U.S. Pat. No. 5,472,412 above;

U.S. Pat. No. 5,711,746: This device is again a rehabilitation device directed to controlling movement for rehabilitation in the rehabilitation process for injured limbs

There are numerous other devices which have been tried to solve this problem. All that I have found follow the same theme of treating or alleviating the pain of tennis elbow by limiting movement, by compression, and the like

To restrain this natural movement of the wrist and other joints in such manner that the full extent of the joint action is curtailed is unnatural and can have lasting adverse affects. Yet, this is what has been done in much of the prior art. In this respect, I consider that there is no true prior art as to a cure, or more correctly prevention and that my invention is unique, novel, and useful.

What is revealed in this patent application is a method and apparatus for preventing, rather than treating, the condition and resulting in a total and natural cure of the problem by avoiding the problem. My approach has apparently never been considered. In this sense, although there has been much development in treating the symptoms, there has not been any direct and effective absolute cure or prevention of the problem, and , thus there is no true prior art.

### SUMMARY OF THE INVENTION

As a physician, frequently I have been consulted for treatment of “tennis elbow” (lateral epicondylitis). This condition is well known to tennis players and others skilled in the art. The customary treatment involves application of splints, braces, supports, application of thermal packs, injections, and the like.

Because of the frequency of such complaints, I made a detailed investigation and physiological analysis of this situation. As a result of much testing and experimentation, and as a result of treatment of patients, and analysis of the muscles, tendons, and ligaments involved, I have discovered the root cause of the ailment commonly referred to as “tennis elbow”, as well as other similar ailments.

I have discovered that the entire condition (and similar conditions in other areas of the body) is centered about the epicondyle area of joints. This is the area located just above the condyle of bones at joints, and which serves generally as an attachment point for muscles, tendons and ligaments which extend to the extremities (hands and feet).

What I discovered was that in a vigorous game of tennis, the player's wrist has the natural tendency to flex forward a great distance after the racket strikes the ball. This is natural, and should occur for normal wrist action. However, the action is so violently forced by the racket momentum, and so sudden, that it puts an undue strain at the lateral upper epicondyle.

I analyzed this with consideration given to movement, momentum, acceleration, and deceleration of the various body parts involved in achieving maximum effectiveness for control of the tennis ball through the action of the tennis racket. I concluded that striking the ball at maximum acceleration was absolutely necessary to the success of a tennis player. I found that virtually instantaneous flexing of the wrist to the maximum and instantaneous return was the natural result. This tendency to resist and to control the wrist and hand is what actually causes the problem at the lateral upper epicondyle. Similar problems can exist for other epicondyle areas such as the medial epicondyle. The intent of my present invention is to eliminate the problem rather than to resist and treat symptoms.

In order to better understand what my invention accomplishes the actual anatomy may be compared to a simple mechanical pulley system where there is an anchor (the lateral upper epicondyle) a cable (the tendon) and another tendon and muscle to the action (the wrist). The primary job of the muscle is to contract. Striking a tennis ball with a tennis racket is not necessarily the primary purpose of the wrist and associated muscles, bones and tendons. It is, in fact, something the wrist was not originally intended for. When the hand holds the tennis racket and causes the racket to impact upon a tennis ball it pulls on the cable and with the muscle trying to contract there is an inordinate amount of stress on the cable and into the bone.

I have now conceived and perfected the solution to the problem. I have developed a method and apparatus which can be worn by a tennis player or other athlete or active person which allows the virtually instantaneous and full force swing of the tennis racket or other item and contact with the ball or the like to achieve maximum acceleration to the ball and allow the full return action of the wrist, but with the final trajectory of the wrist and the return to the position for the next stroke of the racket or the like being tempered in such manner that the undue stress at the lateral upper epicondyle area is eliminated.

As a part of my study and experimentation I have found that I am able to make specific accommodations for persons having some abnormalities related to epicondyle areas which enables them to participate in certain activities which were not possible before this invention.

My invention is not aimed at alleviating the discomfort of tennis elbow. My intent is to prevent the occurrence of tennis elbow. When properly trained using my invention, the wrist eventually becomes “trained” so that it follows the proper full travel after hitting a tennis ball with the racket but it does so in a less violent manner. When this full training is accomplished, the device of this invention may be discarded and the wrist will act in a proper manner and not aggravate the lateral upper epicondyle.

In doing what I have outlined, and which will be further detailed below, I have not “treated” tennis elbow and the like, I have eliminated it.

The method and apparatus described in this patent application is, also, applicable to other ailments associated with epicondyle areas of the skeletal structure, as will be understood by those skilled in the art.

It is an object of this invention to provide a method and apparatus for eliminating lateral upper epicondyle injury and irritation;

It is another object of this invention to provide such a method and apparatus which will not interfere with full and complete activity while engaged in vigorous sports;

Another object of this invention is to provide such a method and apparatus which can be adjusted to meet the special needs of persons having physical abnormalities.

The foregoing and other objects and advantages of this invention will become apparent to those skilled in the art upon reading the description of a preferred embodiment, which follows, in conjunction with a review of the appended drawings.

### BRIEF DESCRIPTION OF THE DRAWINGS

FIG. 1 is a schematic side elevation of a tennis player's hand, with a portion of the forearm and tennis racket handle, after striking a ball;

FIG. 2 is a schematic side elevation of an arm of a person wearing an apparatus suitable to practice the method of this invention;

FIG. 3 is an enlarged schematic sectioned view of the element 40 of FIG. 2; and

FIG. 4 is an enlarged schematic, broken away, perspective of an alternate embodiment of an apparatus suitable to practice the method of this invention.

### DESCRIPTION OF A PREFERRED EMBODIMENT

An inventory of the items bearing reference numerals on the drawings is:

| Numeral | Item |
|---|---|
| 10 | human hand and arm with apparatus to practice the method |
| | of this invention |
| 10a | hand |
| 12 | upper arm |
| 14 | elbow |
| 16 | forearm |
| 17 | thumb |
| 18 | metacarpal area |
| 19 | forefinger |
| 20 | glove |
| 20a | wrist band |
| 21 | wrist |
| 22 | batten |
| 22a | batten |
| 22b | batten joint or flex position |
| 24 | glove armband |
| 30 | tensioning mechanism |
| 32 | cable anchor |
| 33 | anchor post |
| 34 | fulcrum pivot point |
| 35 | fulcrum |
| 36 | cable |
| 36a | weld |
| 37 | cable anchor |
| 38 | cable |
| 38a | weld |
| 39 | anchor post |
| 40 | tension control |
| 42 | cylinder |
| 43 | weld |
| 43a | weld |
| 44 | cylinder end |
| 45 | space around piston |
| 46 | cylinder end |
| 47 | hole in end 46 |
| 48 | coil spring |
| 50 | floating piston |
| 54 | fluid |
| 56 | packing |
| 110 | tennis racket handle |
| 116 | forearm |
| 121 | wrist |
| 122 | hand |
| 124 | thumb |
| 130 | angle between hand and forearm |
| 200 | magnetic wrist control, generally |
| 220 | electro-magnet core |
| 222 | electro-magnet core |
| 236 | cable |
| 236a | cable connection to core |
| 238 | cable |
| 242 | rail |
| 244 | slide mount on rail |
| 246 | fixed mount on rail |
| 250 | electro-magnet |
| 251 | coil housing |
| 252 | electro-magnet |
| 253 | coil housing |
| 254 | coil |
| 256 | coil |
| 260 | control |
| 261 | lead |
| 262 | battery |
| 263 | lead |
| 266 | electrical lead to coil |
| 268 | electrical lead to coil |
| 280 | control |
| 281 | lead |
| 282 | battery |
| 283 | lead |
| 286 | electrical lead to coil |
| 288 | electrical lead to coil |

In normal activities, the human wrist may constantly bend without any undue aggravation of the elbow epicondyle areas. However, as shown in FIG. 1, after striking a tennis ball with racket 110 a typical tennis player's wrist 121 will flex virtually instantaneously and violently beyond normal flexing so that the angular relationship 200 of the hand 122 and thumb 124 to the forearm 116 is approximately ninety degrees or less. The leverage of the tennis racket is so great with a tight grip of the hand on the handle that the wrist is actually pulled to a greater distortion, or minimizing of the angle between the hand and the forearm than nature originally intended. This violent and sudden movement causes the muscles attached to the lateral upper epicondyle to resist the flexing of the wrist leading to injury at the lateral upper epicondyle.

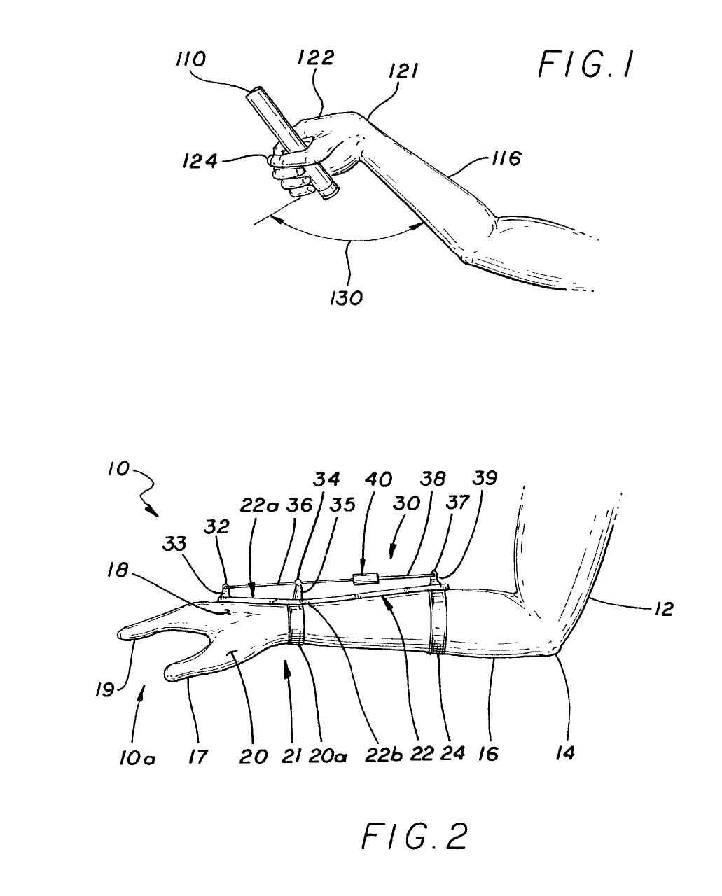

FIG. 2 illustrates one embodiment of an apparatus suitable to practice the method of this invention. A tennis player's arm 10 is shown. The elbow 14 between the upper arm 12 and the forearm 16 is the area where the pain of tennis elbow is felt. I have found that this discomfort is caused by the violent and rapid flexing action of the wrist.

In FIG. 1 the wrist 21, is in a normal position in line between the forearm 16 and the hand 10a. As illustrated in FIG. 1, the hand 10a will be wearing a glove terminating in a wrist band 20a on wrist 21. The glove carries batten(s) 22-22a. This may be a single batten with an area 22b which will allow flexing, or it may be two battens 22 and 22a with the flexing position 22b being a hinge or the like. This will be readily understood by those skilled in the art. The batten 22 will terminate near the elbow with an attached adjustable holding band 24 made with hook and loop fastening material or the like for ease of application and adjustment.

Tension control 40 comprises a cylinder 42 with ends 44 and 46 welded, or otherwise fastened, at 43a and 43. A floating piston 50 is located within the cylinder with clearance 45 sufficient to allow lateral movement within the cylinder and to allow fluid 54, which fills the cylinder, to pass from one side of the piston to the other as the piston moves laterally in the cylinder. Cable 36 is fastened to piston 50 by welding or the like at 36a. Cable 38 is welded or otherwise fastened at 38a to cylinder end 44. Cable 36 passes through fluid tight packing box 56 in opening 47 in the cylinder end 46. Cable 38 is anchored to anchor post 39 fastened to glove 20 and fastening band 24 by clamping or other means known to those skilled in the art. Cable 36 passes through pivot point eye 34 (the eye is not visible on the drawing but it will be understood by those skilled in the art) on fulcrum 35 which is mounted on the batten 22a by means known to those skilled in the art. Cable 36 is clamped or otherwise fastened at 32 to anchor post 33 which is fastened to batten 22a. The cables 38 and 36 are taut when the hand, wrist, and forearm are lineally aligned as shown in FIG. 2. A coil spring or the like 48 is used to maintain the piston closely adjacent cylinder end 44 when the hand, wrist, and forearm are in the position shown in FIG. 2.

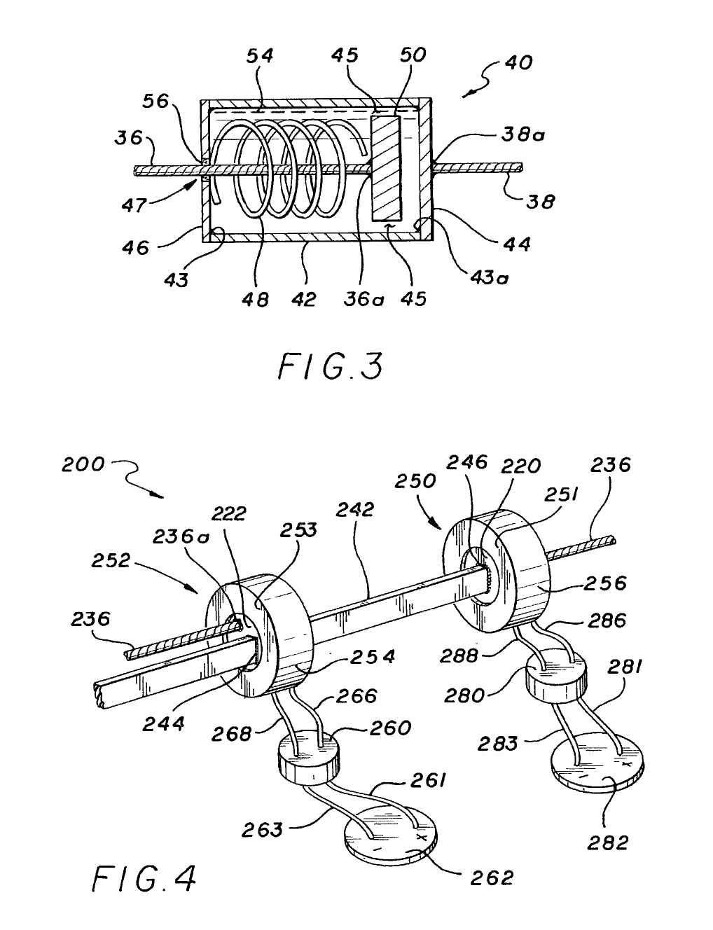

After hitting a ball, when the wrist commences its travel to the position shown in FIG. 3 the cable 36 will pull on the piston. The piston will move slowly as fluid 54 flows slowly around the piston through the space 45. The rate of travel is adjusted to individual requirements by adjusting the fluid viscosity, adjusting the space 45 about the cylinder, and adjusting the compression of spring 54.

The result is that the wrist will travel to the full extent as shown in FIG. 3, but it will travel at a controlled speed and acceleration. The wrist will not travel in its unfettered violent motion. Thus, the violent aggravation of the elbow epicondyle will not occur.

FIG. 4 illustrates an alternate embodiment of the tension control. In this case, the overall mechanism is essentially the same as shown in FIG. 2. The difference in the overall mechanism as compared to FIG. 2 lies in the tension control, itself. In this case I use two electro-magnets 250 and 252 to control the tension.

Electro-magnet 250 will be fixed upon non-magnetic rail 242 which will be mounted on a base element associated with the battens in the glove of FIG. 2 in a manner known to those skilled in the art. Electro-magnet 252 will be mounted on bearings or the like (not shown, but understood by those skilled in the art) so that it can slide lineally on the rail 242. In this manner, the tension on cable 236 (comparable to cable 36 in FIG. 2) can be increased or decreased by increasing or decreasing the magnetic attraction of the two electro-magnets.

Electro-magnet 250 comprises core 220 and coil 256 within coil housing 251. Electrical power is obtained by coil 256 from battery or the like 282 through leads 286 and 288 from control unit 280, which is powered by battery 282. Electro-magnet 252 comprises core 222 and coil 254 within coil housing 253. Electrical power is obtained by coil 254 from battery or the like 262 through leads 266 and 268 from control unit 260, which is powered by battery 262. In this embodiment I use what amounts to a mini-computer (not shown, but understood by those skilled in the art) as the control 260 and/or 280. The mini-computers will constantly adjust the electrical power being fed to the coils so as to vary the magnetic attraction between the two electro magnets in response to the wrist motion to achieve the exact most desired restraint and control of wrist motion.

In addition to the exact embodiments shown to practice the method of this invention, I have developed additional features which will be understood by those skilled in the art. For example, In the case of the fluid control of the embodiment of FIG. 2, I am able to control the flow of fluid past the piston in the embodiment shown by an individually programmed semi-conductor activated iris diaphragm or the like. In each case, there even can be an individual algorithm prepared, as will be understood by those skilled in the art.

Many other modifications may be made to the physical and operating configurations of devices to practice the method of this invention without departing from the essential underling principle of the invention which is controlling wrist action in order to eliminate the violent action normally encountered in many sports such as tennis, other racket sports, golf, and the like.

By this reference I incorporate the claims and abstract, which follow, within this description of a preferred embodiment the same as though they were fully set forth again.

While the embodiments of this invention shown and described are fully capable of achieving the objects and advantages desired, it is to be understood that such embodiments are for purposes of illustration only, and not for purposes of limitation.

In the claims which follow, I may fail to claim a patentable feature of this invention. In that event, such failure to claim a feature will be due to inadvertence and mistake. In the event I should fail to claim a patentable feature it is not my intention to dedicate or abandon it. In that event, upon discovering any such failure to claim a patentable feature I shall immediately seek to claim such feature by any appropriate means such as re-issue or the like.

I claim:

1. The method for avoiding epicondyle aggravation (tennis elbow) including: equipping a person engaging in a game of tennis normally resulting in epicondyle aggravation with a device which resiliently tempers the rate, but not the extent, of movement of a hand and wrist affecting an epicondyle in such manner that full and complete movement of the hand and wrist takes place in a non-violent and natural manner, relieving the epicondyle and associated tendons, muscles, and ligaments from the stress of untempered violent movement of the hand and wrist.

2. The method of claim 1 wherein the device includes an electro-mechanical device.

3. The method of claim 1 wherein the device includes an hydraulic device.

4. The method of avoiding epicondyle aggravation including: equipping a person engaged in strenuous activity normally resulting in epicondyle aggravation with a programable device programed to resiliently temper the rate, but not the extent, of movement of a hand and wrist affecting an epicondyle according to an algorithm in such manner that the epicondyle and associated tendons, muscles, and ligaments are relieved from the affect of non-programed violent movement of the extremity.

5. The method of claim 4 wherein the program of the programable device is such as to cause the hand and wrist concerned to react in a manner which does not aggravate the epicondyle concerned.

6. The method of claim 4 wherein an individualized algorithm is the program for the programable device.

7. Apparatus for preventing epicondyle aggravation in a human engaged in strenuous physical activity comprising: means suitable to affect the otherwise violent movement of a hand and wrist of the human while so engaged associated with the hand and wrist in such manner that the otherwise violent motion becomes a natural, non-violent, full and complete movement of the hand and wrist.

8. The apparatus of claim 7 wherein the means suitable to affect the otherwise violent movement includes an algorithm to prevent violent movement.

9. The apparatus of claim 8 wherein the means to convert the otherwise violent movement includes programable means suitable to perform an individualized algorithm which causes the otherwise violent movement to become non-violent full, complete, and natural movement of the hand and wrist.
